(19) United States
(12) Patent Application Publication
(10) Pub. No.: US 2026/0194464 A1
(43) Pub. Date: Jul. 9, 2026

(54) NON-INVASIVE IMAGING SYSTEM FOR IMAGING BIOLOGICAL MATERIALS

(71) Applicant: MONASH UNIVERSITY, Clayton (AU)

(72) Inventors: Fabrizzio Enrique Horta Nunez, Clayton (AU); Adrian Neild, Clayton (AU); Victor Javier Cadarso Busto, Clayton (AU); Reza Nosrati, Clayton (AU); Erick Javier Vargas Ordaz, Clayton (AU); Mohammad Hossein Haft Tananian, Clayton (AU); Alex De Marco, Clayton (AU); Sergey Gorelick, Clayton (AU)

(21) Appl. No.: 19/128,824

(22) PCT Filed: Nov. 10, 2023
(86) PCT No.: PCT/AU2023/051132

§ 371 Date: May 9, 2025

(30) Foreign Application Priority Data
Nov. 11, 2022 (AU) 2022903395

(51) Int. Cl. G01N 21/64; G01N 21/01

(52) U.S. Cl. CPC G01N 21/6458; G01N 21/6486; G01N 2021/0112; G01N 2021/6421; G01N 2021/6478; G01N 2021/6482; G01N 2021/6484; G01N 2201/1042

(57) ABSTRACT

Described herein is an imaging device (100) adapted to be incorporated into a device (200) for containing biological material (102). The imaging device (100) includes a sample holder (104) configured to hold a sample of the biological material (102). An input (106) is configured for receiving a beam of light (108). An illumination system (110) configured to convert the beam of light (108) into a two-dimensional sheet of light (112) and for directing the sheet of light (112) onto a target illumination zone (114). A transport mechanism (116) is adapted to move the target illumination zone (114) relative to the sample holder (104) such that the sheet of light (112) passes across the sample to illuminate the biological material (102). An imaging system (116) is positioned to receive at least a portion of the light returned from the biological material (102) and to direct the returned light onto an image sensor (120) to generate a plurality of images of the sample obtained at different positions of the sheet of light across the sample.

### FIELD OF THE INVENTION

The present application relates to imaging biological material and in particular to imaging live gametes and/or embryos.

Embodiments of the present invention are particularly adapted for performing non-invasive light sheet fluorescence microscopy on live gametes or embryos. However, it will be appreciated that the invention is applicable in broader contexts and other applications such as non-fluorescence imaging.

### BACKGROUND

Assisted reproductive technologies (ARTs) have been developing in the last four decades. The success of ART can be improved by transferring multiple embryos. However, this technique comes with additional cost and complications associated with an increased risk of multiple pregnancies.

More recently, selecting a single embryo with the highest probability of yielding a live birth has been an alternative strategy for improving the success rate of assisted reproduction. Current techniques for selecting embryos either have limited success and/or can damage the embryos themselves.

Therefore, there is a critical need for alternative methods that can accurately assess embryo quality without any potential adverse effects on the embryo's integrity to directly guide the selection process.

Selecting the most suitable embryos for implantation is crucial to the success rate of assisted reproduction and offspring health. Besides morphological evaluation using optical microscopy, a promising alternative is the non-invasive imaging of live embryos to establish metabolic performance. Embryo metabolism plays a key role during the early developmental stages as significant metabolic changes take place during the first days after fertilization.

However, assessing embryos' mitochondrial metabolic status has been only achieved using state-of-the-art fluorescence microscopy with methods that are costly and challenging, thereby limiting the potential for deployment within fertility clinics. For example, microscopy including fluorescence lifetime imaging microscopy (FLIM) and hyperspectral microscopy have been implemented for assessing embryo viability. However, both FLIM and hyperspectral microscopy are expensive and highly complex in operation while the laser excitation could also result in phototoxicity that may hamper embryo viability.

Any discussion of the background art throughout the specification should in no way be considered as an admission that such art is widely known or forms part of common general knowledge in the field.

### SUMMARY OF THE INVENTION

In accordance with a first aspect of the present invention, there is provided an imaging device adapted to be incorporated into a device for containing biological material, the imaging device including:
a sample holder configured to hold a sample of the biological material;
an input for receiving a beam of light;
an illumination system configured to convert the beam of light into a two-dimensional sheet of light and for directing the sheet of light onto a target illumination zone;
a transport mechanism adapted to move the target illumination zone relative to the sample holder such that the sheet of light passes across the sample to illuminate the biological material; and
an imaging system positioned to receive at least a portion of the light returned from the biological material and to direct the returned light onto an image sensor to generate a plurality of images of the sample obtained at different positions of the sheet of light across the sample.

In some embodiments, the biological material includes one or more gametes or embryos and the device for culturing biological material includes an incubator for incubating the one or more gametes or embryos.

In some embodiments, the light returned from the biological material includes light fluoresced from the biological material. In some embodiments, the light returned from the biological material includes light emitted due to autofluorescence from the one or more gametes or embryos.

In some embodiments, the transport mechanism includes a first actuator adapted to selectively move one or more microlenses within the illumination system such that the target illumination zone moves across the sample holder. In some embodiments, the transport mechanism includes a second actuator adapted to move one or more microlenses within the imaging system in conjunction with the first actuator. The first and/or second actuators may include a motorised or movable stage. In some embodiments, the first and second actuators include a single motorised stage configured to move the illumination system and imaging system as one.

In some embodiments, the sample holder includes a microfluidic channel and the transport mechanism includes a microfluidic system configured to move the sample along the microfluidic channel through the illumination zone such that the one or more gametes or embryos are passed through the sheet of light.

In some embodiments, the transport mechanism includes an actuator configured to move the sample holder such that the one or more gametes or embryos are passed through the sheet of light.

In some embodiments, the illumination system and the imaging system are formed from a monolithic structure.

In some embodiments, the one or more gametes or embryos are unstained.

In some embodiments, the imaging system has a numerical aperture of greater than or equal to 1.

In some embodiments, the input includes an optical fibre.

In some embodiments, the illumination system is configured to generate the sheet of light in a substantially horizontal plane. In other embodiments, the illumination system is configured to generate the sheet of light in a substantially vertical plane.

In some embodiments, the illumination system includes a single cylindrical microlens.

In some embodiments, the beam of light has a wavelength in the range of 400 nm to 850 nm.

Preferably the illumination system and imaging system are micro-optical systems formed of smaller scale components than a conventional benchtop optical system.

In some embodiments, the imaging system is adapted to generate one or more multispectral images of the sample across a plurality of different wavelengths.

In accordance with a second aspect of the present invention, there is provided a method of imaging biological material when located in a device for containing biological material, the method including:
receiving a beam of light from an input;
positioning an illumination system to convert the beam of light into a two-dimensional sheet of light and for directing the sheet of light onto a target illumination zone;
moving the target illumination zone relative to a sample holder which is holding a sample of the biological material such that the sheet of light passes across the sample to illuminate the biological material; and
positioning an imaging system to receive at least a portion of the light returned from the biological material and to direct the returned light onto an image sensor to generate a plurality of images of the biological material obtained at different positions of the sheet of light across the sample.

### BRIEF DESCRIPTION OF THE FIGURES

Example embodiments of the disclosure will now be described, by way of example only, with reference to the accompanying drawings in which:

FIG. 1 is a schematic sideview of an imaging device for imaging a biological material that is incorporated into a device for containing the biological material;

FIG. 2 is a schematic system diagram of a device for containing biological material including an imaging device;

FIG. 3 is a schematic elevated perspective view of a first embodiment of the imaging device of FIG. 1;

FIG. 4 is a schematic elevated perspective view of a second embodiment of the imaging device of FIG. 1;

FIG. 5 is a schematic plan view of a third embodiment of the imaging device of FIG. 1;

FIG. 6 is an expanded view of an illumination region of the third embodiment of FIG. 5;

FIG. 7 is a flow diagram illustrating the primary steps in a method of imaging biological material when it is located in a device for containing biological material such as an incubator;

FIG. 8, panel (a) is a colour photograph showing the polydimethylsiloxane (PDMS) device setup;

FIG. 8, panel (b) is a schematic of the optofluidic device concept showing the coupling of the IVF pipette tips in the inlet of the microchannel;

FIG. 8, panel (c) a microscopic photograph of the device illustrating three 2-cell mouse embryos traveling from the IVF pipette tip to the microchannel and panel;

FIG. 8, panel (d) is a microscopic photograph of the device showing 2-cell mouse embryos passing the light-sheet;

FIG. 9, panel (a) illustrates NAD(P)H autofluorescence images of different sections of a 2-cell embryo using the optofluidic device of FIG. 8, the sequence shows a cross-section of the mouse embryo every 6.6 μm (a subset of the ones collected every 0.45 μm).

FIG. 9, panel (b) is a reconstructed 3D image of the NAD(P)H signal using a full sequence of images showing the spatial distribution of the NAD(P)H in the blastomere 1 and blastomere 2;

FIG. 9, panel (c) is an image of a maximum internal projection of the full sequence of images;

FIG. 10, panel (a) is a schematic of the scanning area of the imaging device of FIG. 8 where embryos travel in the microchannel at constant speed to cross the light-sheet. The white lines indicate streamlines in the microfluidic channel;

FIG. 10, panel (b) is a superimposed experimental image of a focused laser beam showing where the light-sheet is formed in the zoomed image of the microchannel in the imaging device of FIG. 8. The light-sheet at the focus has a thickness of 1.8 μm (FWHM in the y-axis) and a height of 75 μm (FWHM in the z-axis), therefore, the area of major intensity is 135 μm²;

FIG. 10, panel (c) is a heat map displaying the exposure dose as a function of flow velocity and laser power. Doses greater than 50 J cm⁻² are indicated with dots. The stars indicate the optimal doses used in our experiments, labelled as high-dose (16 J cm⁻²) and low-dose (8 J cm⁻²);

FIG. 11, panel (a) is a comparison of the signal to noise ratio (SNR) of raw NAD(P)H fluorescent signals between a Maximum Intensity Projection (MIP) image obtained using confocal fluorescent microscopy and the optofluidic device;

FIG. 11, panel (b) an MIP image captured at high-dose power, 16 J cm⁻²;

FIG. 11, panel (c) an MIP image captured at low-dose power, 8 J cm⁻²;

FIG. 11, panel (d) illustrates the intensity profile of line L1 in FIGS. 11(a) to (c) compared with the intensity profile of the background (bg line);

FIG. 11, panel (e) illustrates the intensity profile of line L2 in FIGS. 11(a) to (c) compared with the intensity profile of the background (bg line);

FIG. 11, panel (f) illustrates the intensity profile of line L3 in FIGS. 11(a) to (c) compared with the intensity profile of the background (bg line);

FIG. 11, panel (g) illustrates the intensity profile of line L4 in FIGS. 11(a) to (c) compared with the intensity profile of the background (bg line);

FIG. 11, panel (h) illustrates the intensity profile of line L5 in FIGS. 11(a) to (c) compared with the intensity profile of the background (bg line);

FIG. 12, panel (a) illustrates a reconstructed 3D image of a blastocyst mouse embryo cultured without the inhibitor treatment (control sample) using the microfluidic system of FIGS. 5 and 6;

FIG. 12, panel (b) illustrates a reconstructed 3D image of an early blastocyst mouse embryo cultured with the inhibitor (FK866) treatment (Inhibitor sample) using the microfluidic system of FIGS. 5 and 6;

FIG. 12, panel (c) illustrates a plot line of the intensity distribution of every image of the stack recorded from the autofluorescence signal of blastocyst embryos without the inhibiting treatment (top curve) and from embryos with the inhibiting treatment (bottom curve;

FIG. 12, panel (d) illustrates a box plot of the intensity distribution of the samples control and inhibitor; and

FIG. 13 illustrates the total number of embryos reaching the blastocyst stage for each condition (control, sham and illuminated) for a high-dose setting (16) cm⁻², n=30) experiment.

### DESCRIPTION OF THE INVENTION

Embodiments of the present invention are particularly adapted for imaging biological material in the form of gametes or embryos in a non-invasive environment so as to be able to gain information about gamete/embryo metabolism and genetic integrity. However, it will be appreciated that the present invention is applicable in broader contexts to imaging other types of biological materials.

### System Overview

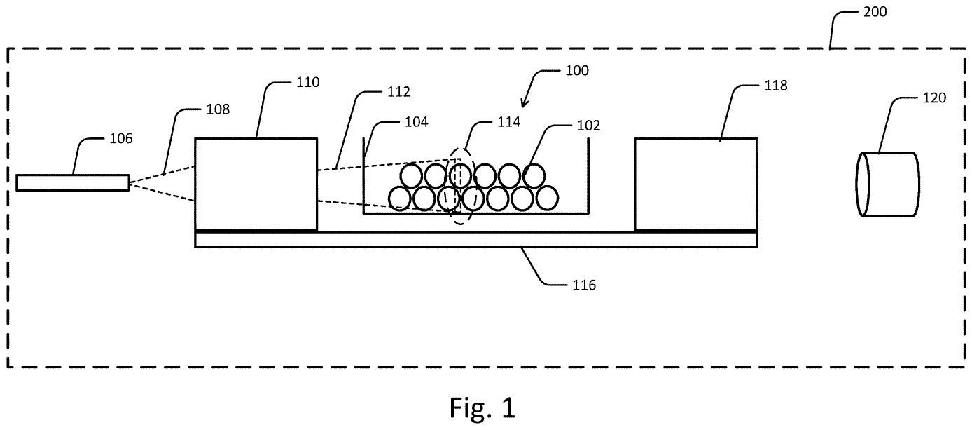

Referring initially to FIG. 1, there is illustrated an imaging device 100 adapted to be incorporated into a device 200 for containing biological material 102. Device 200 is preferably a benchtop or portable incubator device that is adapted for storing, maintaining and culturing the biological material under controlled conditions such that the biological material 102 is not damaged. By way of example, where the biological material 102 includes gametes or embryos, device 200 may be an incubator device configured to incubate or culture the gametes or embryos at a temperature of about 37° C. with about 5% CO₂.

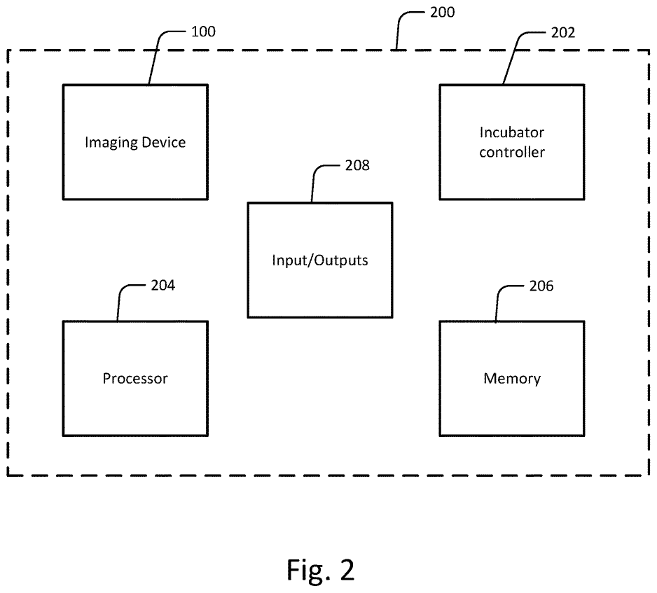

As shown in FIG. 2, device 200 includes the imaging device 100 as well as various other elements such as a controller 202, processor 204, memory 206 and inputs/outputs 208. Controller 202 is adapted for controlling various elements of device 200 such as temperature, climate and movement of elements within imaging device 100 as described below. Processor 204 is adapted for processing images captured by imaging device 100 such as to generate three dimensional images from a plurality of two-dimensional images. Memory 206 is adapted for storing data including image data from imaging device 100 and other data relevant to the culturing of the biological material. Device 200 also includes inputs/outputs 208 in the form of user interfaces (e.g. a touchscreen display), network ports, power cables and wireless network controllers (e.g. Wi-Fi device) for communicating with external devices.

Referring again to FIG. 1, the imaging device 100 includes a sample holder 104 configured to hold a sample of the biological material 102. The sample holder 104 may be in the form of microwell, cuvette or capillary being either sealed to define an internal enclosed environment or having one or more openings such that the biological material 102 is at least partially exposed to an environment within device 200.

Imaging device 100 also includes an input 106 for receiving a beam of light 108. Input 106 may be in the form of an optical fibre or optical fibre connector adapted to receive an optical fibre. The optical fibre or other input is adapted to either generate or propagate light from a source of light such as a laser to produce beam of light 108. The light source may have a single narrow linewidth comprising a central wavelength or may comprise a broad range of wavelengths. In some embodiments, the light source may include a tunable laser or multiple light sources having different spectral profiles.

The laser source preferably emits electromagnetic radiation in the range of wavelengths from 400 nm to 850 nm. However, emission of radiation at around 405 nm and 468 nm have been found to be particularly advantageous for illuminating embryos in a non-invasive manner to initiate autofluorescence.

By way of example, a suitable laser operating at 405 nm is a Fabry-Perot fibre-coupled laser source (Thorlabs, New Jersey, USA. Part Number: S3FC405), which can be connected to input 106 in the form of a single-mode optical fibre (Thorlabs, New Jersey, USA. Part Number: P 1-405B-FC).

An illumination micro-optical system 110 is configured to convert the beam of light 108 into a thin sheet of light 112 and for directing the sheet of light 112 onto a target illumination zone 114. System 110 is termed a “micro-optical system” as it contains smaller scale components than a conventional benchtop optical system. This includes components such as microlenses and microprisms with physical dimensions typically in the range of a couple of millimetres. However, it will be appreciated that larger scale components may be used in micro-optical system 110 such that it can be referred to as a conventional imaging system.

The sheet of light 112 is formed by focusing a beam of light in only one dimension by a cylindrical lens or similar optical element to create a highly elliptical beam profile. The sheet of light 112 has a thickness across the thin focused axis that is typically in the order of nanometres or microns and this is used to illuminate a thin slice of the sample.

A transport mechanism 116 is adapted to move the target illumination zone 114 relative to the sample holder 104 such that the sheet of light 112 passes across the sample to illuminate the biological material. The term “relative” is used to mean that the target illumination zone 114 and/or the sample holder 104 may be moved relative to each other. In the embodiment illustrated in FIG. 1, the target illumination zone 114 is moved while the sample holder 104 and biological material 102 is maintained stationary. This has the advantage of reducing potential damage to the biological material 102 during movement. In other embodiments, the sample holder 104 is moved while the target illumination zone 114 is maintained stationary.

An imaging micro-optical system 118 is positioned to receive at least a portion of the light returned from the biological material 102 and direct the returned light onto an image sensor 120 to generate a plurality of images of the sample obtained at different positions of the sheet of light across the sample. Depending on the biological material 102 being imaged and the particular application, the returned light may represent reflected, backscattered, fluoresced or autofluouresced light from the sample. System 118 is also termed a “micro-optical system” as it contains smaller scale components than a conventional benchtop optical system. This includes components such as microlenses and microprisms with physical dimensions typically in the range of a couple of millimetres. However, it will be appreciated that larger scale components may be used in micro-optical system 118.

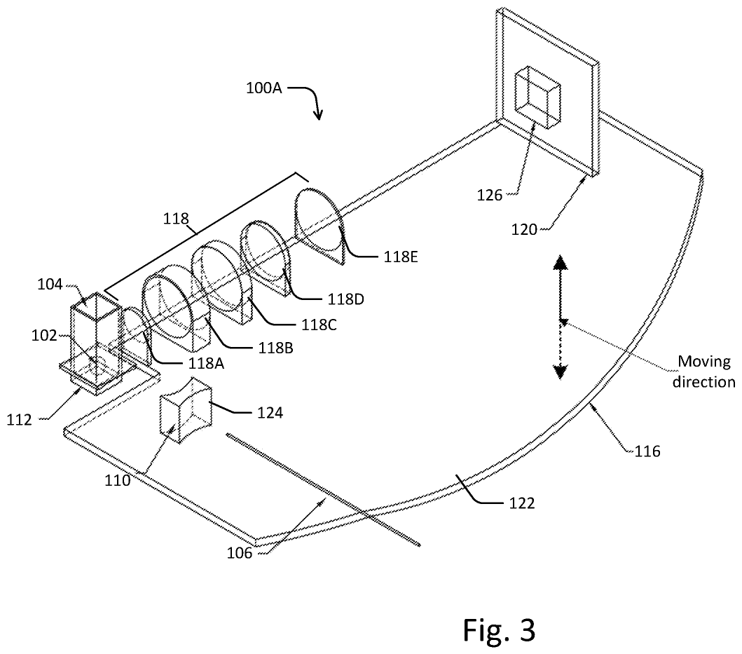

Referring to FIG. 3, a first embodiment imaging system 100A is illustrated. Corresponding features from the imaging system 100 of FIG. 1 are designated with like reference numerals. In the imaging system 100A, the transport mechanism 116 includes an actuator (not shown) configured to selectively adjust the position of a moving stage 122. The actuator may include a mechanical or motorised device such as a screw actuator or may include a piezoelectric device. Moving stage 122 is adapted to selectively vertically move a cylindrical microlens 124 within the illumination micro-optical system 110, as well as the imaging micro-optical system 118, input 106 and image sensor 120, such that the target illumination zone 114 and sheet of light 112 move vertically across the sample holder 104 and biological sample 102. The sheet of light 112 generated by cylindrical microlens 124 is substantially horizontally planar so as to illuminate horizontal slices of biological sample 102. The vertical thickness of the sheet of light 112 is preferably in the range of a few hundred nanometers to a few microns. During this movement, a portion of the light returned from the biological sample 102 at each vertical position of moving stage 122 is directed along an imaging path through the imaging micro-optical system 118 and is imaged at a sensor array 126 of image sensor 120. In other embodiments, the sheet of light 112 generated by cylindrical microlens 124 is substantially vertically planar so as to illuminate vertical slices of biological sample 102.

In the imaging system 100A, the imaging micro-optical system 118 includes a series of lenses 118A-118E to shape and focus the returned light onto sensor array 126 in the manner of a microscope objective. However, it will be appreciated that the imaging micro-optical system may include other numbers and configurations of optical elements, including lenses, mirrors, prisms etc. Further, in the imaging system 100A, the illumination micro-optical system 110, the imaging micro-optical system 118 and input 106 are each mounted onto moving stage 122 so as to move vertically together when the position of moving stage 122 is adjusted. Moving stage 122 may be controlled by incubator controller 202 of device 200 (see FIG. 2) or by a separate controller. The vertical movement of moving stage 122 facilitates the imaging of horizontal slices of the biological material 102 at each position of the stage.

The resulting stack of two-dimensional images can be combined by processor 204 to generate one or more three-dimensional images of the biological material such as a fluorescence image in the case of auto fluorescent material. The thickness of the sheet of light 112 and the relative speed of the sample holder 104 relative to the sheet of light 112 determines, at least in part, the resolution of the resulting images. Other factors such as the numerical aperture of the detection objective may also define the image resolution.

In some embodiments, a multispectral image may be obtained by coupling multiple light sources or a tunable light source through input 106 and performing multiple passes of sheet of light 112 across biological material 102 in a single imaging session. Alternatively, multiple wavelengths of light may be coincident onto illumination zone 114 at any instant in time such that a multispectral image may be generated from a single pass of sheet of light 112 across biological material 102.

In imaging system 100A, the illumination path, including input 106 and cylindrical microlens 124 is positioned at right angles to the imaging path, including imaging micro-optical system 118 and image sensor 120. This is a configuration for light sheet fluorescence microscopy (LSFM) systems to improve the signal to noise ratio. In the case where the biological material 102 includes one or more gametes or embryos, illumination at a wavelength such as 405 nm can instigate autofluorescence and some of the light from this process can be directed through the imaging micro-optical system 118 and captured by image sensor 120 to generate a fluorescence image of the sample.

Although in imaging system 100A each of the input 106, illumination cylindrical microlens 124 and imaging micro-optical system 118 and image sensor 120 are moved in conjunction with moving stage 122, it will be appreciated that other configurations are possible. In some embodiments, only a subset of the components is moved during the imaging. By way of example, in one embodiment, the transport mechanism includes an actuator adapted to move one or more microlenses within the illumination micro-optical system 110 and/or the input 106 without moving the imaging micro-optical system 118 or image sensor 120. In other embodiments, two separate actuators are employed; one to selectively move the illumination micro-optical system 110 and input 106 and another to selectively move the imaging micro-optical system 118 and image sensor 120.

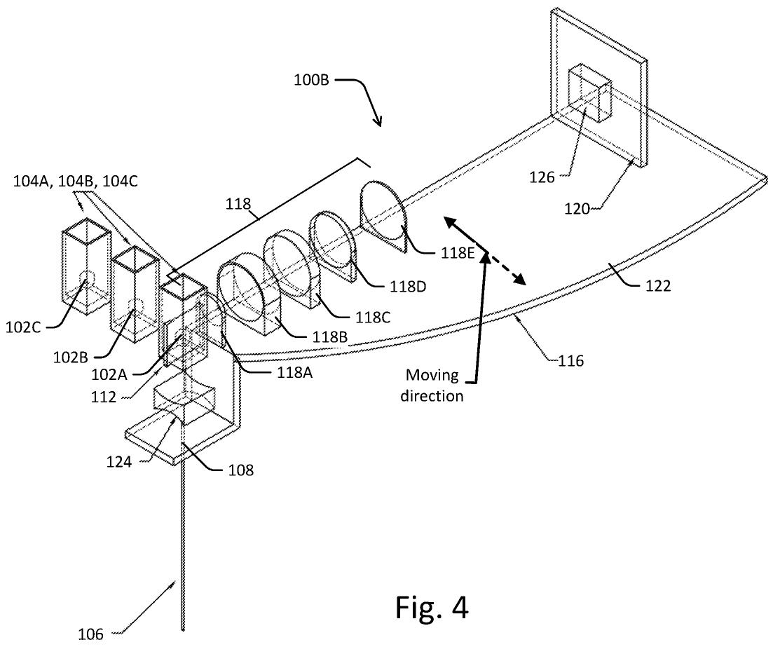

FIG. 4 illustrates a further embodiment imaging system 100B that is similar in operation to that of imaging system 100A but with components oriented vertically. In particular, input 106 is disposed substantially vertically to direct beam of light 108 vertically upward through illumination cylindrical microlens 124 to generate a substantially vertical sheet of light 112. The moving stage 122 is configured to slideably move horizontally to move input 106, microlens 124, imaging micro-optical system 118 and image sensor 120 (each of which are mounted to the moving stage 122) in conjunction with each other. This horizontal movement allows the substantially vertical sheet of light 112 to be progressively scanned across a plurality of sample holders in the form of micro wells 104A-104C. Each micro well contains a respective biological sample in the form of embryos 102A-102C. The setup of imaging system 100B allows multiple biological samples to be imaged without manual intervention by an operator.

In further embodiments (not shown), the transport mechanism 116 includes one or more actuators configured to move the sample holder 104 such that the biological material 102 (e.g. one or more gametes or embryos) is passed horizontally through the sheet of light 112.

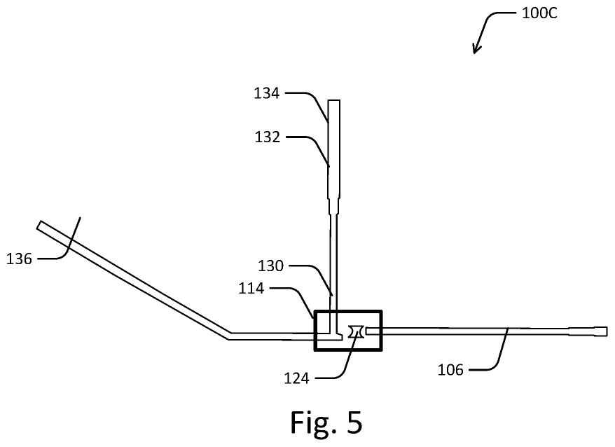

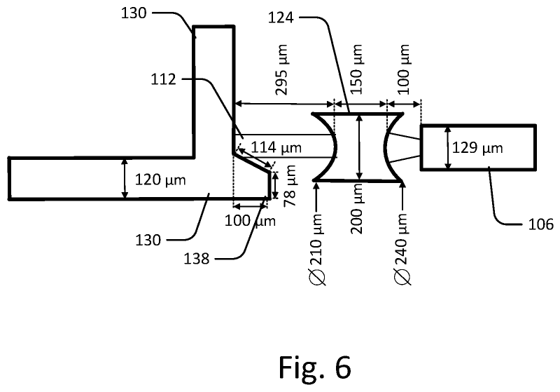

Referring now to FIGS. 5 and 6, there is illustrated a further embodiment imaging system 100C incorporating a microfluidic channel for moving the biological material 102 while maintaining the illumination micro-optical system 110 and imaging micro-optical system 118 stationary. In this embodiment, the sample holder 104 includes a microfluidic channel 130 and the transport mechanism 116 includes a microfluidic system 132. The microfluidic system 132 is configured to move the sample in a fluid along the microfluidic channel 130 from an input 134 through the illumination zone 114 to an output 136 such that the one or more gametes or embryos are passed through the sheet of light 112.

FIG. 6 illustrates a close up of the imaging system 100C around the illumination zone 114. Example dimensions and characteristics are shown. The microchannel has a width of about 120 μm while the cylindrical microlens 124 produces a sheet of light at the centre of the microchannel 130 between 1.8 μm to 3 μm. The sheet of light has a thickness of 114 μm in this embodiment.

The illumination micro-optical system 110 includes a single cylindrical microlens 124. As illustrated, the microchannel 130 includes a corner 138 where the imaging occurs. The corner 138 is designed at a sharp protrusion corner configuration to avoid optical aberrations due to the index reflections miss matching. Additionally, for tracking the sample at all times when travelling in the microchannel 130 and safely retrieving it, the configurations of the inlet 134 and outlet 134 are oriented horizontally so as to integrate IVF micropipette tips to the ports.

In the imaging system 100C of FIGS. 5 and 6, each of the components forming the illumination micro-optical system 110, imaging micro-optical system 118 and microfluidic system 132 may be formed monolithically by etching from a single substrate material. The integrated micro-optical components are pre-aligned to the microfluidic channel 130 used to deliver the samples. The micro-optical components are cast directly in polydimethylsiloxane (PDMS).

Micro-optical components will normally render high aberrations and low numerical aperture. The imaging system 100C overcomes this by combining micro-optical elements with a microfluidic system that allows the manipulation of the sample in a self-aligned fashion without the need of moving parts or alignment while keeping the distance between all the components in the microscopic range. The imaging system is sufficiently efficient to perform imaging on unstained samples, which produce an autofluorescence signal that is orders of magnitude lower than that of stained samples.

In the embodiments illustrated and described above, both the illumination micro-optical system 110 and the imaging micro-optical system 118 are formed from a monolithic structure such as a PDMS substrate.

In each of the embodiments described above, the imaging micro-optical system 118 is able to produce a numerical aperture of greater than or equal to 1. This allows efficient coupling to image gametes or embryos are unstained. In some embodiments, the micro-optical construct is monolithic, self-aligned and able to produce a light-sheet narrow enough to work with an objective with an NA of 1.05 or more.

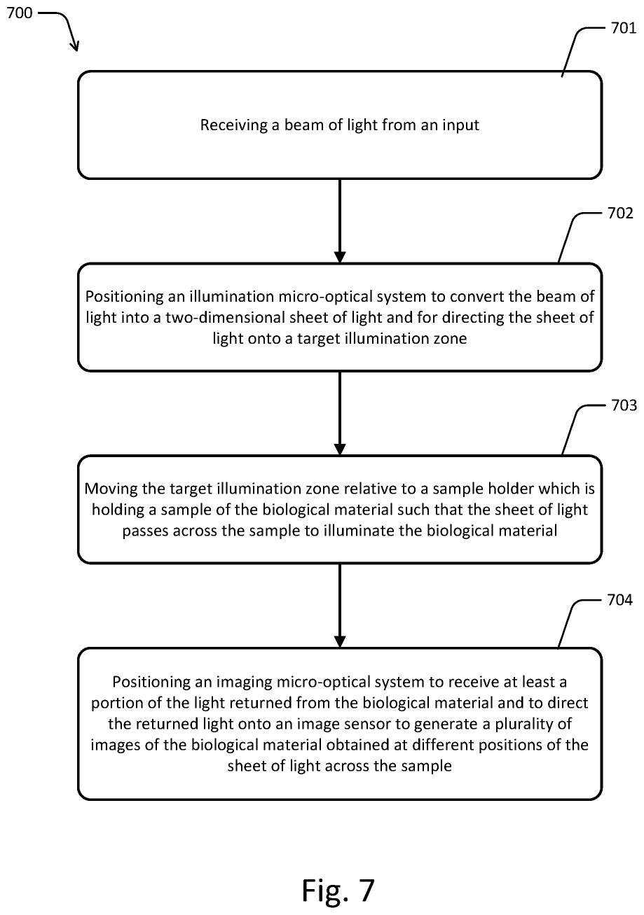

Referring to FIG. 7, the systems and devices described above are adapted to perform a method 700 of imaging biological material 102 contained in a storage device. The method includes, at step 701, receiving a beam of light 108 from an input 106. At step 702, an illumination micro-optical system 110 is positioned to convert the beam of light 108 into a two-dimensional sheet of light 112 and for directing the sheet of light 112 onto a target illumination zone 114. At step 703, the target illumination zone 114 is moved relative to a sample holder 104, which is holding a sample of the biological material 102 such that the sheet of light 112 passes across the sample to illuminate the biological material 102. At step 704, an imaging micro-optical system 118 is positioned to receive at least a portion of the light returned from the biological material and to direct the returned light onto an image sensor 120 to generate a plurality of images of the biological material 102 obtained at different positions of the sheet of light 112 across the sample.

### Example—Microfluidics Embryo Delivery

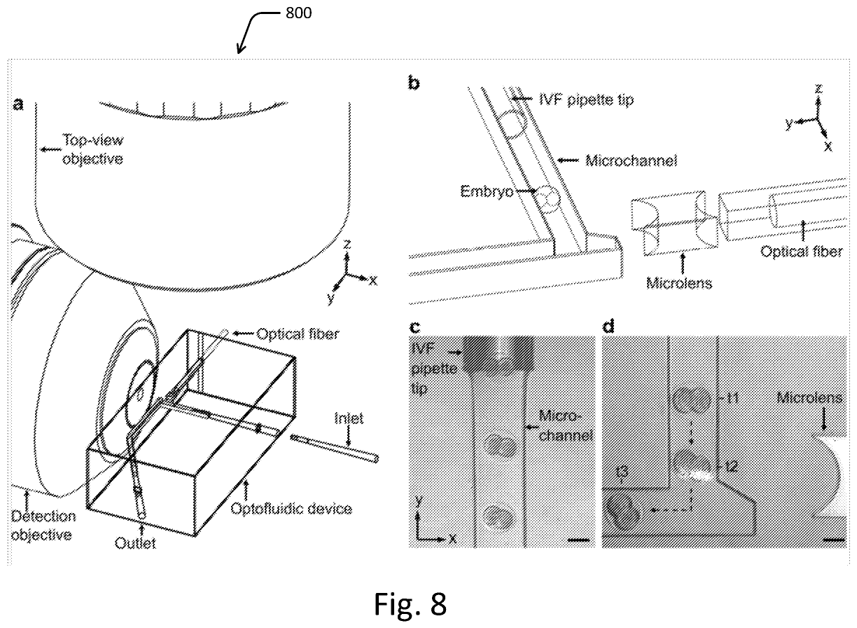

An example implementation of the invention is described below which uses a microfluidic system to move two-cell mouse embryos through an imaging system in a similar manner to that illustrated in FIGS. 5 and 6. The imaging system 800 is illustrated schematically in FIG. 8. Panel (a) is a colour photograph showing the PDMS fabricated device setup, panel (b) is a schematic of the optofluidic device concept showing the coupling of the IVF pipette tips in the inlet of the microchannel, panel (c) is a microscopic photograph of the device illustrating three 2-cell mouse embryos traveling from the IVF pipette tip to the microchannel and panel (d) is a microscopic photograph of the device showing 2-cell mouse embryos passing the light-sheet.

Imaging device 800 is a scalable and powerful optofluidic device is provided, which is capable of obtaining 3D images of a nicotinamide adenine dinucleotide phosphate (NAD(P)H) signal of live early-stage mouse embryos via LSFM. This optofluidic approach provides a high signal-to-noise ratio (SNR) by using a low light dose at an excitation wavelength of 405 nm. The device 800 provides a well-designed fluidic environment to allow for safe handling of mouse embryos as they pass in and out of the light-sheet generated on-chip at the center of the microchannel. The non-invasive nature of the method is demonstrated by evaluating the viability and the development of illuminated embryos as compared with non-illuminated embryos. This optofluidic method provides a promising opportunity for real-time analysis of embryo quality in infertility clinics to potentially achieve improved reproductive outcomes, without inducing phototoxicity or damaging the embryo.

The imaging device 800 and petri dish are mounted on a Peltier module in the outlet for keeping both the imaging device and petri dish at about 37° C. A heat incandescent lamp (not shown in FIG. 8) was implemented as well to maintain the system at 37° C. As illustrated in FIG. 8, panel (b), the NAD(P)H imaging is performed via LSFM. Forming of the sheet of light is performed on-chip with a microlens and an optical fibre and recording of the fluorescent signal is performed off-chip with an objective lens.

The optical system in imaging device 800 was designed to obtain the emission for NAD(P)H measurements using a 1.05 NA detection objective (Olympus, Tokyo, Japan. Part Number: UPLSAPO 30XS) using a blue fluorescent protein bandpass range filter [430-490] nm (Thorlabs, New Jersey, USA. Part Number: MF 460-60), an infinity-corrected tube lens (Thorlabs, New Jersey, USA. Part Number: TTL180-A) and a CMOS camera (Basler AG, Ahrensburg, Germany. ITEM #acA1920-155 μm-Basler ace). The optical system was mounted on the XYZ translation stage (Thorlabs, New Jersey, USA. Part Number: T1220D) placed on an optical table (Thorlabs, New Jersey, USA. Part Number: T1220D) using a rail system (Qioptiq, Rhyl, UK. X 95 Profile System). The sensor of the camera was set to have a binning factor of 2 horizontally and vertically making the final pixel size of 0.39 μm for the optical detection system.

A top-view system was used to place the optical fibre into the device as well to locate the mouse embryos when travelling into the device. The optical system was integrated by a dry long working distance 5× objective (Thorlabs, New Jersey, USA. Part Number: MY5X-802), a fixed tube lens of 160 mm (EHD imaging GmbH, Damme, Germany. Part Number: FT160), a LED light source (EHD imaging GmbH, Damme, Germany. Part Number: IL100), and a CMOS camera (Basler AG, Ahrensburg, Germany. Part Number: acA1920-155 μm-Basler ace). In order to block and avoid light noise on the detection objective, a fluorescent filter was placed in the LED light source (Thorlabs, New Jersey, USA. Part Number: MF 535-22). This optic system was assembled on different XYZ translation stages (Thorlabs, New Jersey, USA. Part Number: T1220D) placed onto a similar rail system (Qioptiq, Rhyl, UK. X 95 Profile System) on the same optical table used in the section 4.1.1 of this paper. The final pixel size of this top-view microscope was 1.465 μm.

The optofluidic device was fabricated out of PDMS by a single step UV lithography, which creates smooth mirror-like and near vertical inner sidewalls, and was capable of handling live two-cell mouse embryos for the purpose of obtaining 3D images of their autofluorescence NAD(P)H signal. The design of the imaging device 800 was tailored from previous work by the inventors (see reference 3) to safely image early-stage mouse embryos. Specifically, the inlet and outlets were redesigned such that they utilised in vitro fertilization (IVF) pipette tips (see FIG. 8, panels (a) and (b)) integrated into the PDMS to facilitate sample handling. This feature allowed a top-view camera to continuously track the location of the embryos whilst in the chip, and assisted in post imaging retrieval (See FIG. 8, panels (c) and 8(d)). Furthermore, the system was held at 37° C. for the duration of the imaging to provide a physiologically relevant environment. After imaging, all embryos were collected to assess their viability, development and quality.

A low-pressure syringe pump (Cetoni GmbH, Korbussen, Germany. ITEM #NEM-B101-03 A) was used to work with low flow regimes using a PEEK tubing connector glass syringe of a volume of 500 μL (SETonic GmbH, Ilmenau, Germany. Part Number: 3010236). In order to connect the IVF pipette tip for loading the embryos (MXL3-125, The Stripper, CooperSurgical Fertility Solutions, Denmark) to the syringe, an adapter of rubber tubing, 0.5 mm ID·1.3 mm OD (Gecko Optical Scientific Equipment, Western Australia, Australia. Part Number: 310 0504) was connected to a PTFE tubing, 0.012″ ID·0.030″ OD, (John Morris Group, Victoria, Australia, ITEM #06417-11) assemble the pipette tip and the syringe together. An integrated heat incandescent lamp (Philips InfraRed Industrial Heat Incandescent Lamp PAR38 IR 100W 240V Red E27) was carefully positioned to have 37° C. and keep the media inside the syringe warm.

In order to load the embryos from their tissue culture plate to the optofluidic device the pipette tip was assembled to the syringe controlling the flow with the syringe pump. The pipette was manually placed on the culture plate, and used a USB microscope for carefully selecting the embryos (ViTiny, Microlinks Technology Corp., Taiwan R. O. C. Part Number: UM12).

NAD(P)H autofluorescence excitation was achieved by exposure to a sheet of light with a wavelength of 405 nm. A 405 nm Fabry-Perot fibre-coupled laser source (Thorlabs, New Jersey, USA. Part Number: S3FC405) was connected to single-mode optical fibre (Thorlabs, New Jersey, USA. Part Number: P1-405B-FC). The second end of the optical fibre was cut by a fibre cleaver (Thorlabs, New Jersey, USA. Part Number: XL411). In order to carefully position the cleaved fibre tip into the optofluidic device, the fibre was placed onto a tapered V-Groove Fibre Holder (Thorlabs, New Jersey, USA. Part Number: HFV002) on a 3-axis manual stage (Thorlabs, New Jersey, USA. Part Number: MAX313D/M).

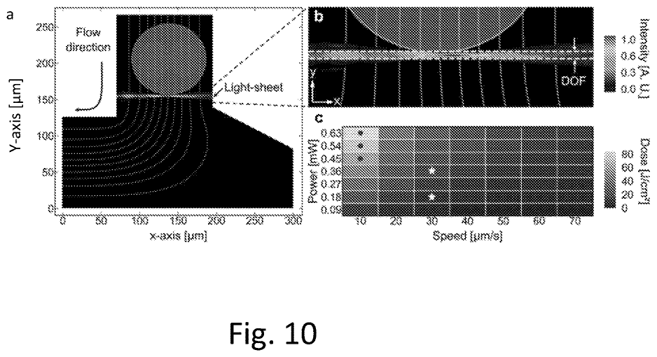

The 405 nm laser light coming out from an optical fibre was focused by a set of cylindrical micro-lenses to create a light sheet across the width of the channel. The laser power was therefore distributed across each cross-section of the light sheet (yz planes in FIG. 10(b)).

The light-sheet dimensions were 1.8 μm in thickness (FWHM in the y-axis) and 75 μm in height (FWHM in the z-axis), therefore, the area of major intensity was 135 μm² (See FIG. 10, panel (b)). Using this, the laser density distribution in the light-sheet was calculated at different laser powers to obtain the laser dose at which the embryos were exposed when crossing the light sheet at different speeds. The heatmap in FIG. 10, panel (c) indicates the exposure dose as a function of laser power and embryo speed. Two doses well below 50 J·cm⁻² were selected to ensure that any potential photodamage is minimized (3-fold and 6-fold smaller), while still achieving a high signal-to-noise ratio (SNR) and high-quality imaging. These were achieved by fixing the speed at 30 μm s⁻¹ and only modifying the laser power, corresponding to 0.36 mW for a dose of 16 J cm⁻² (high-dose) and 0.18 mW for 8 J cm⁻² (low-dose).

Avoiding the use of light in the ultraviolet range (between 100 and 400 nm) reduces the risk of damaging the embryos during illumination, but reduces the efficiency of fluorescence excitation of the optimal excitation at 340 nm. To overcome this drawback, a high NA silicon immersion microscope objective (NA=1.05) was used to increase the amount of fluorescent light being captured. Furthermore, the 800 μm working distance of the objective dictated the proximity of the microfluidic channel to the edge of the chip which it was carefully designed to avoid silicon oil dripping. Additionally, silicone immersion oil (refractive index, RI=1.40) was used to reduce spherical aberrations by bridging the refractive index mismatch between the cells (intracellular RI=1.38) 55 and the PDMS (5:1 PDMS ratio RI=1.41) 56.

As a high NA objective obtains light over a wide angle, the geometry of the microchannel's corner where the imaging occurs was designed with a sharp protrusion (See FIG. 8, panel (d)) to avoid any lateral change on the RI before the light is collected, preventing optical aberrations. A factor to be considered to avoid affecting the quality of the acquired images is that when loading multiple embryos, they need to be separated at least 200 μm (two embryos length) between each other. If not, the embryo upstream (t3 in FIG. 8, panel (d)) will create aberrations for the next embryo being imaged (t2 in FIG. 8, panel (d)). This will happen as the fluorescent light of t2 will go through t3 before the images are captured.

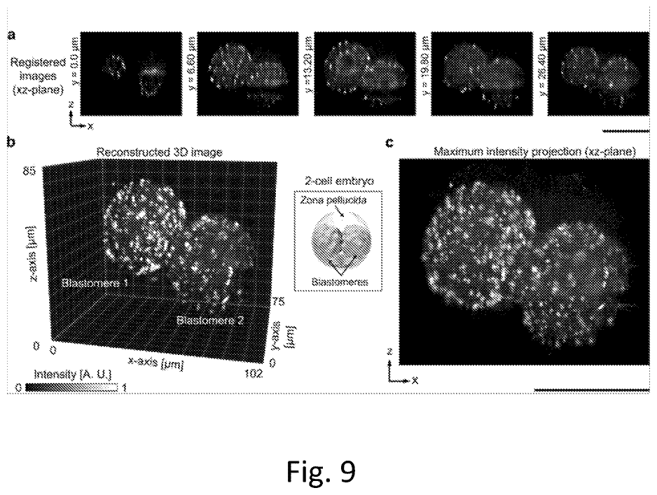

These combined improvements resulted in high contrast fluorescent images of the embryos, as shown in FIG. 9. By application of a fluid flow along the channel, the embryos were carried through the sheet of light whilst images were recorded, allowing a stack of cross-sectional images to be captured. Through the use of a top view camera (see FIG. 8, panel (a)), which records the embryo's speed of the passage through the light-sheet, a volumetric image can be reconstructed using the stack of images collected.

Compared to prior art systems, the configuration of the imaging device 800 of FIG. 8 allows a more versatile recording of microscopy-grade image quality (i.e. high spatial resolution and high SNR) when using a high-NA objective (NA>1) as the physical limitation of the orthogonal geometry was eliminated allowing a wider range of working distances objectives. Single-objective LSFM is achieved by removing the excitation objective and using a micro-mirror that reflects and focuses the light-sheet at the center of the microchannel.

When the two-cell embryos passed through the sheet of light (see FIG. 8, panel (c)), biomolecules within their two blastomeres are excited by the photons from the 405 nm laser source, emitting an autofluorescence signal from the NADH and NADPH in its mitochondria and cytoplasm (see FIG. 9). However, as the NADH signal concentration is several folds stronger than the NADPH signal, the convention of referring to this fluorescence signal as only coming from the NAD(P)H biomolecules has been followed. The autofluorescence signal of NAD(P)H comes from two different sources in the embryo. The blur part in FIG. 9, panel (c) belongs to the cytoplasmatic NAD(P)H whereas the clear part belongs to mitochondrial NAD(P)H. As NAD(P)H is highly concentrated in the mitochondria relative to the cytoplasm the mitochondrial NAD(P)H the autofluorescence signal is more intense.

In order to decrease the risk of phototoxicity, the three-dimensional images of the 2-cell embryos were obtained in less than 2 seconds. The specimens crossed the light-sheet in the microchannel at a constant speed of about 30 μm s⁻¹, being transported at low flow rate regimes of 0.01-0.02 μL min⁻¹. When the flow speed fluctuates, e.g. due the existence of air-bubbles, the axial sampling gets compromised. An increase of 10% of the optimal embryo speed (30 μm s⁻¹) results in 5% of less cross-sectional images, which has a negligible effect on the image quality.

The thickness of a cross-sectional image was defined by the thickness of the light-sheet, whereas the axial resolution only depends on the detection objective NA. In the present described optofluidic system, the light-sheet thickness is 1.8 μm at the FWHM and the axial resolution is 1.04 μm (the detection objective's NA=1.05). Although having a thicker light-sheet than the axial resolution provides lower image contrast, it has been associated with improving the axial resolution. In the present case, the theoretical axial resolution was improved by 20%. As a result, high contrast fluorescence images were obtained every 0.45 μm at 66.67 frames per second. Importantly, operating the system at that speed and frame rate avoided under-sampling for three-dimensional imaging mouse embryos.

The full stack of images generated by the two-cell embryo was converted to a volumetric image (see FIG. 9, panel (b)). It is noteworthy that NAD(P)H is highly concentrated in the mitochondria and cytoplasm, and 3D images of the NAD(P)H signal indicated the normal spatial distribution of the mitochondria between blastomeres of the embryo, which it has been also shown with similar technologies such FLIM. Furthermore, recognising the live spatial distribution of NAD(P)H in embryos during embryo development could allow to further understand its relationship with conventional embryo morphology and to determine normal and abnormal mitochondria distribution across the different stages of early embryo development. Including the NAD(P)H signal as a decisive factor in the clinical workflow could potentially open new avenues to also select the highest quality embryos based on their metabolic activity for a higher chance of reaching a clinical pregnancy and live birth in IVF patients.

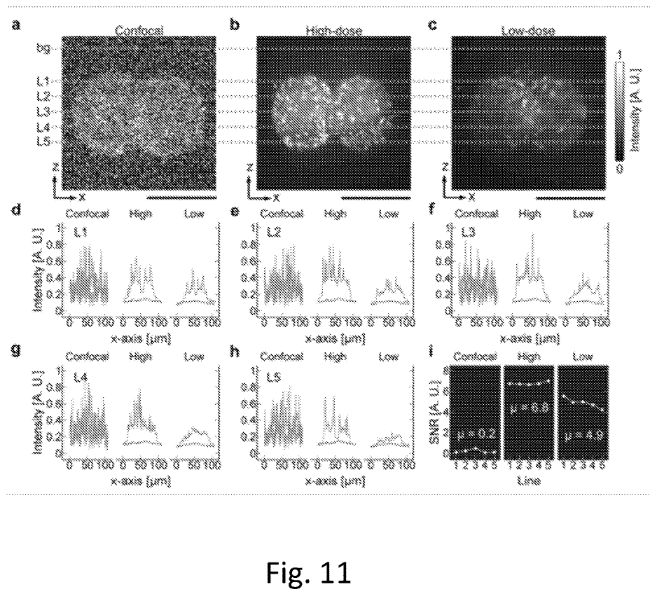

The trade-off of using low excitation doses is a lower intensity image. Thus, the quality of the obtained images was assessed by means of the SNR and this was compared to NAD(P)H images obtained using confocal microscopy. Maximum intensity projection (MIP) images are presented in FIG. 11, panels (a)-(c). To compare the image quality, the intensity profile of five lines (L1 to L5) was obtained equally distributed across the embryo images. These intensity profiles were compared with the intensity profile of the background (bg) for each image (FIG. 4d-h). The SNR was calculated as the difference of the average intensity over the standard deviation between the line profile and the background line.

On average the SNR at low-power for the imaging device 800 was 24.5 times higher (see FIG. 11, panel (i), p<0.00001; t-test) than that obtained with the CFM, while for high-power the SNR was 34 times higher (see FIG. 4(i), p<0.00001; t-test). The results demonstrate that imaging device 800 is capable of detecting the NAD(P)H autofluorescence signal at excitation below those known to cause damage, with a SNR and overall image quality superior to that of the images obtained using traditional confocal microscopy.

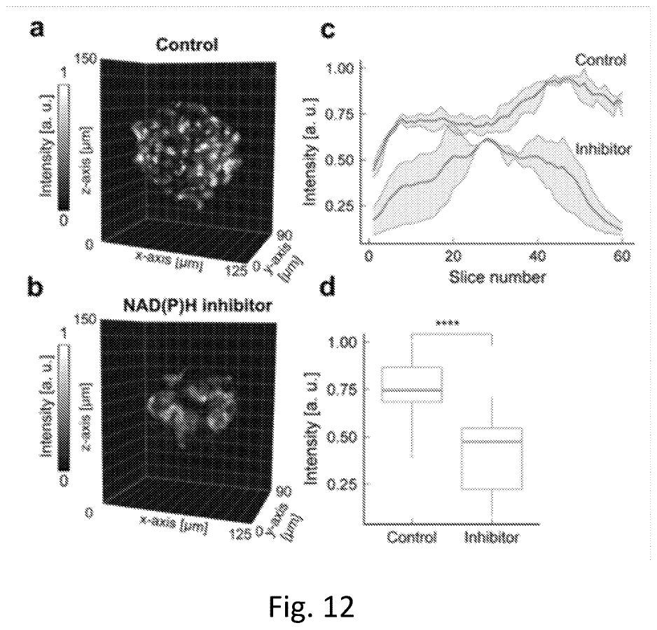

In one experiment, a total of 34 embryos were cultured from 2-cell to blastocyst stage over 3.5 days in an off-chip incubator. Half of the embryos were treated with FK866 to inhibit their metabolic activity, showing a 47% reduction in auto-fluorescence signal compared to the non-treated ones. The results of this experiment are illustrated FIG. 12. FIG. 12, panel (a) illustrates a reconstructed 3D image of a blastocyst mouse embryo cultured without the inhibitor treatment (control sample). The 3D image shows the spatial distribution of the NAD(P)H and was reconstructed using full sequence of images.

FIG. 12, panel (b) illustrates a reconstructed 3D image of an early blastocyst mouse embryo cultured with the inhibitor (FK866) treatment (Inhibitor sample). The 3D image shows the spatial distribution of the NAD(P)H and was reconstructed using full sequence of images.

FIG. 12, panel (c) illustrates a plot line of the intensity distribution of every image of the stack recorded (total of 60 images) from the autofluorescence signal of blastocyst embryos without the inhibiting treatment (top curve) and from embryos with the inhibiting treatment (bottom curve). The bold lines represent the mean intensity, and the grey ribbon depicts the range of the intensity values in each sample. Control group showed a 47% higher NAD(P)H autofluorescence signal than Inhibitor sample counterparts.

FIG. 12, panel (d) illustrates a box plot of the intensity distribution of the samples control and inhibitor which difference is statically significance (p<0.0001; t-test). The results of FIG. 12 validate the use of the above described optofluidic device to image the autofluorescence of NAD(P)H and assess embryos metabolic activity.

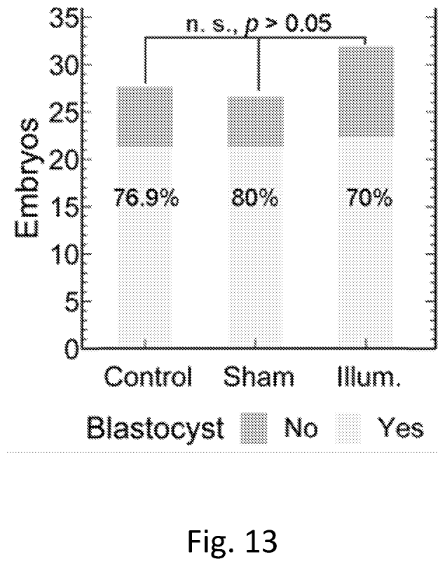

Finally, the embryo viability was studied. Embryos exposed to low and high dose power settings were collected and cultured until the blastocyst stage to analyse their viability, development, and quality post exposure. FIG. 13 illustrates the total number of embryos reaching the blastocyst stage for each condition (control, sham and illuminated) for a high-dose setting (16 J cm⁻², n=30) experiment.

To the best the inventors' knowledge, there has not been a development of an optofluidic device capable of safely handling living mouse embryos to load them and recover them back for the assessment of the embryo viability after being imaged in the optofluidic device.

The invention described above is adapted for the application of monitoring live gametes and early embryos in a non-invasive manner. In particular, it is capable of generating and detecting auto-fluorescence of an embryo without damage.

The system is simple and small enough for integration into a conventional IVF incubator, and yet is capable of layer-by-layer images of embryos/gametes in a time lapsed manner. At the same time, the micro-imaging setup can potentially reduce the overall cost of the system by orders of magnitude when compared to conventional macro-optics imaging devices.

### REFERENCES

The following is a list of references, the contents of which are incorporated herein by way of cross-reference.
1. Sanchez T, Zhang M, Needleman D, Seli E. Metabolic imaging via fluorescence lifetime imaging microscopy for egg and embryo assessment. Fertil Steril 111, 212-218 (2019).
2. McLennan H J, Saini A, Dunning K R, Thompson J G. Oocyte and embryo evaluation by AI and multi-spectral auto-fluorescence imaging: Livestock embryology needs to catch-up to clinical practice. Theriogenology 150, 255-262 (2020).
3. Vargas-Ordaz E J, et al. Three-dimensional imaging on a chip using optofluidics light-sheet fluorescence microscopy. Lab Chip 21, 2945-2954 (2021).
4. Memeo R, et al. Automatic imaging of Drosophila embryos with light sheet fluorescence microscopy on chip. J Biophotonics 14, e202000396 (2021).
5. Sala F, et al. High-throughput 3D imaging of single cells with light-sheet fluorescence microscopy on chip. Biomed Opt Express 11, 4397-4407 (2020).

### INTERPRETATION

Unless specifically stated otherwise, as apparent from the following discussions, it is appreciated that throughout the specification discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “controller” or “processor” may refer to any device or portion of a device that processes electronic data, e.g., from registers and/or memory to transform that electronic data into other electronic data that, e.g., may be stored in registers and/or memory. A “computer” or a “computing machine” or a “computing platform” may include one or more processors.

Reference throughout this specification to “one embodiment”, “some embodiments” or “an embodiment” means that a particular feature, structure or characteristic described in connection with the embodiment is included in at least one embodiment of the present disclosure. Thus, appearances of the phrases “in one embodiment”, “in some embodiments” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

It should be appreciated that in the above description of exemplary embodiments of the disclosure, various features of the disclosure are sometimes grouped together in a single embodiment, Fig., or description thereof for the purpose of streamlining the disclosure and aiding in the understanding of one or more of the various inventive aspects. This method of disclosure, however, is not to be interpreted as reflecting an intention that the claims require more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

Embodiments described herein are intended to cover any adaptations or variations of the present invention. Although the present invention has been described and explained in terms of particular exemplary embodiments, one skilled in the art will realize that additional embodiments can be readily envisioned that are within the scope of the present invention.

1. An imaging device adapted to be incorporated into a device for containing biological material, the imaging device including:
a sample holder configured to hold a sample of the biological material;
an input for receiving a beam of light;
an illumination system configured to convert the beam of light into a two-dimensional sheet of light and for directing the sheet of light onto a target illumination zone;
a transport mechanism adapted to move the target illumination zone relative to the sample holder such that the sheet of light passes across the sample to illuminate the biological material; and
an imaging system positioned to receive at least a portion of the light returned from the biological material and to direct the returned light onto an image sensor to generate a plurality of images of the sample obtained at different positions of the sheet of light across the sample.

2. The imaging device according to claim 1 wherein the biological material includes one or more gametes or embryos and the device for culturing biological material includes an incubator for incubating the one or more gametes or embryos.

3. The imaging device according to claim 1 wherein the light returned from the biological material includes light fluoresced from the biological material.

4. The imaging device according to claim 1 wherein the light returned from the biological material includes light emitted due to autofluorescence from the one or more gametes or embryos.

5. The imaging device according to claim 1 wherein the transport mechanism includes a first actuator adapted to selectively move one or more microlenses within the illumination system such that the target illumination zone moves across the sample holder.

6. The imaging device according to claim 5 wherein the transport mechanism includes a second actuator adapted to move one or more microlenses within the imaging system in conjunction with the first actuator.

7. (canceled)

8. The imaging device according to claim 6 wherein the first and second actuators include a single motorised stage configured to move the illumination system and imaging system as one.

9. The imaging device according to claim 1 wherein the sample holder includes a microfluidic channel and the transport mechanism includes a microfluidic system configured to move the sample along the microfluidic channel through the illumination zone such that the one or more gametes or embryos are passed through the sheet of light.

10. The imaging device according to claim 1 wherein the transport mechanism includes an actuator configured to move the sample holder such that the one or more gametes or embryos are passed through the sheet of light.

11. The imaging device according to claim 1 wherein the illumination system and the imaging system are formed from a monolithic structure.

12. The imaging device according to claim 2 wherein the one or more gametes or embryos are unstained.

13. The imaging device according to claim 1 wherein the imaging system has a numerical aperture of greater than or equal to 1.

14. The imaging device according to claim 1 wherein the input includes an optical fibre.

15. The imaging device according to claim 1 wherein the illumination system is configured to generate the sheet of light in a substantially horizontal plane.

16. The imaging device according to claim 1 wherein the illumination system is configured to generate the sheet of light in a substantially vertical plane.

17. The imaging device according to claim 1 wherein the illumination system includes a single cylindrical microlens.

18. The imaging device according to claim 1 wherein the beam of light has a wavelength in the range of 400 nm to 850 nm.

19. The imaging device according to claim 1 wherein the imaging system is adapted to generate one or more multispectral images of the sample across a plurality of different wavelengths.

20. The imaging device according to claim 1 wherein the imaging system and illumination system are micro-optical systems.

21. A method of imaging biological material when located in a device for containing biological material, the method including:
receiving a beam of light from an input;
positioning an illumination system to convert the beam of light into a two-dimensional sheet of light and for directing the sheet of light onto a target illumination zone;
moving the target illumination zone relative to a sample holder which is holding a sample of the biological material such that the sheet of light passes across the sample to illuminate the biological material; and
positioning an imaging system to receive at least a portion of the light returned from the biological material and to direct the returned light onto an image sensor to generate a plurality of images of the biological material obtained at different positions of the sheet of light across the sample.
